(19) United States
(12) Patent Application Publication
(10) Pub. No.: US 2016/0038550 A1
(43) Pub. Date: Feb. 11, 2016

(54) METHODS AND COMPOSITIONS FOR TREATING CONDITIONS ASSOCIATED WITH MEMORY LOSS

(71) Applicant: Craig E. Kinzer, Seattle, WA (US)

(72) Inventor: Craig E. Kinzer, Seattle, WA (US)

(21) Appl. No.: 14/805,455

(22) Filed: Jul. 21, 2015

Related U.S. Application Data
(60) Provisional application No. 62/027,391, filed on Jul. 22, 2014.

(51) Int. Cl. A61K 36/16; A61K 31/343; A61K 31/353; A61K 31/192

(52) U.S. Cl. CPC A61K 36/16; A61K 31/192; A61K 31/343; A61K 31/353

(57) ABSTRACT

Methods and compositions are described for treating, preventing, ameliorating, or the like one or more symptoms associated with memory loss. Also described are pharmaceutical compositions including one or more of a monoamine oxidase inhibitor, a cyclooxygenase inhibitor, a nootropic agent, a terpene, a terpenoid, and a pharmaceutically acceptable carrier.

### CROSS-REFERENCE TO RELATED APPLICATION

This application claims the benefit under 35 U.S.C. §119(e) of U.S. Provisional Patent Application No. 62/027,391 filed Jul. 22, 2014. This provisional application is incorporated herein by reference in its entirety.

### SUMMARY

In an aspect, the present disclosure is directed to, among other things, a pharmaceutical composition including a monoamine oxidase inhibitor, a cyclooxygenase inhibitor, and a pharmaceutically acceptable carrier.

In an aspect, the present disclosure is directed to, among other things, a pharmaceutical composition including at least one nootropic agent, at least one cyclooxygenase inhibitor, and a pharmaceutically acceptable carrier.

In an aspect, the present disclosure is directed to, among other things, a pharmaceutical composition including a monoamine oxidase inhibitor, a cyclooxygenase inhibitor, a terpene, and a pharmaceutically acceptable carrier.

In an aspect, the present disclosure is directed to, among other things, a pharmaceutical composition including a terpenoid, a cyclooxygenase inhibitor, and a pharmaceutically acceptable carrier.

In an aspect, the present disclosure is directed to, among other things, a method of preventing or treating a condition associated with memory loss. In an embodiment, the method includes administering to a mammal in need thereof a therapeutically effective amount of a cyclooxygenase inhibitor in combination with a monoamine oxidase inhibitor.

In an embodiment, the method includes administering to a mammal in need thereof a pharmaceutically active amount of a cyclooxygenase inhibitor in combination with a monoamine oxidase inhibitor, and a nootropic agent.

In an aspect, the present disclosure is directed to, among other things, a method of treating, preventing, or ameliorating one or more symptoms associated with memory loss. In an embodiment, the method includes administering to a subject in need thereof a therapeutically effective amount of a cyclooxygenase inhibitor in combination with a nootropic agent.

In an aspect, the present disclosure is directed to, among other things, a method of preventing or treating a condition associated with a loss of a cognitive ability in a human subject. In an embodiment, the method includes administering to a subject in need thereof a therapeutically effective amount of a cyclooxygenase inhibitor in combination with at least one terpene and at least one terpenoid.

The foregoing summary is illustrative only and is not intended to be in any way limiting. In addition to the illustrative aspects, embodiments, and features described above, further aspects, embodiments, and features will become apparent by reference to the drawings and the following detailed description.

### BRIEF DESCRIPTION OF THE DRAWINGS

FIG. 1 is a schematic diagram of a signaling cascades elicited downstream of CB receptor activation by endocannabinoids and cannabinoids according to an embodiment.

### DETAILED DESCRIPTION

Cannabinoid receptors are part of the cannabinoid receptor system in the brain and are involved in a variety of physiological processes including nociception (pain sensation), appetite, lipid metabolism, gastrointestinal motility, cardiovascular modulation, motor activity, mood, and memory. See e.g., Panagiotis et al., The Neuroprotective Role of Endocannabinoids against Chemical-induced Injury and Other Adverse Effects. Journal of Applied Toxicology 33.4: 246-64 Web (2013) (which is incorporated herein by reference). In an embodiment, cannabinoids, cannabidiols, cannabinols, and the like extracted from Cannabis sativa L, may act at peripheral sites and yield analgesia through the action on CB1 and CB2 receptors. See e.g., Jorge et al., J. Pain Res.; 4:11-24. doi: 10.2147/JPR.S9492 (December 2010) (which is incorporated herein by reference). In an embodiment, cannabidiols may have anxiolytic effects both in humans and in animals. See e.g., Bergamaschi et al., Neuropsychopharmacology, 36(6): 1219-1226. doi: 10.1038/npp.2011.6 (May 2011) (which is incorporated herein by reference). In an embodiment, cannabinoids may be effective in treating chemotherapy-induced emesis. See e.g., Williamson et al., Cannabinoids in Clinical Practice, Drugs, 60(6):1303-14 (December 2000).

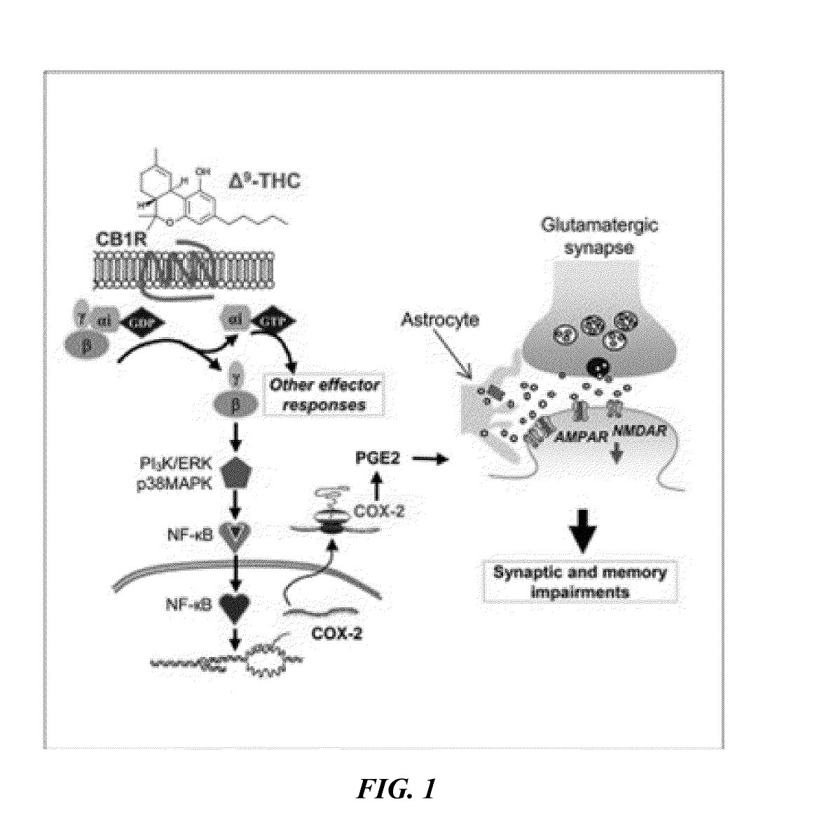

Δ⁹-tetrahydrocannabinol, a cannabinoid, may increase the levels of cyclooxygenase-2 (COX-2) in the hippocampus. Δ9 THC may induce COX-2 expression and activity via CB1 receptor-coupled G protein βγ subunits. See, e.g., Chen et al., Δ9 THC-Caused Synaptic and Memory Impairments Are Mediated through COX-2 Signaling, CELL, 155:1154-65 (Nov. 21, 2013) (which is incorporated herein by reference). Referring to FIG. 1, impaired synaptic and cognitive function induced by repeated Δ9 THC exposure may be associated with a CB1R-Gβγ-Akt-PI3K/ERK/p38MAPK-NF-kB-COX-2 signaling pathway. See e.g., Chen et al., Δ⁹-THC-caused synaptic and memory impairments are mediated through COX-2 signaling, Cell 155:1154-1165 (Nov. 21, 2013) (which is incorporated herein by reference).

Synaptic impairment, memory impairment, and the like in a human being may be mediated through COX-2 signaling. For example, pharmacological inhibition of COX-2 may block downregulation and internalization of glutamate receptor subunits and alterations of the dendritic spine density of hippocampal neurons induced by repeated Δ⁹-tetrahydrocannabinol exposures. Pharmacological inhibition of COX-2 may also reduce Δ⁹-tetrahydrocannabinol-impaired hippocampal long-term synaptic plasticity, spatial learning, and memory.

In an embodiment, methods and compositions include at least one monoamine oxidase inhibitor in combination with at least one COX-2 inhibitor in the manufacture of a pharmaceutical formulation for use in the prevention or treatment of a condition associated with memory loss. In an embodiment, a pharmaceutical composition includes at least one monoamine oxidase inhibitor, at least one cyclooxygenase inhibitor, and a pharmaceutically acceptable carrier.

In an embodiment, a pharmaceutical composition includes at least one monoamine oxidase inhibitor. In an embodiment, the monoamine oxidase inhibitor comprises a reversible monoamine oxidase inhibitor. Non-limiting examples of monoamine oxidase inhibitors include one or more extracts from banisteriopsis caapi, ginkgo biloba, passiflora, pausinystalia johimbe, peganum harmala, Hypericum perforatum (e.g., St John's Wort), or the like. Further non-limiting examples of monoamine oxidase inhibitors include curcumin, harmaline, harmine, kavalactones, myristicin, or the like. Further non-limiting examples of monoamine oxidase inhibitors include furazolidone, isocarboxazid, L-deprenyl, moclobemide, pargyline, phenelzine, tranylcypromine, or the like.

In an embodiment, the monoamine oxidase inhibitor comprises at least one flavonoid. Non-limiting examples of flavonoids include anthocyanidins, anthoxanthins, flavanones, flavanonols, flavans, isoflavonoids, or the like. Further non-limiting examples of flavonoids include comprises apigenin, quercetin, cannflavin A, β-sitosterol, or the like. In an embodiment, the at least one flavonoid comprises apigenin, quercetin, cannflavin A, or β-sitosterol.

In an embodiment, the monoamine oxidase inhibitor comprises one or more ginkgo biloba plant extracts. For example, in an embodiment, the monoamine oxidase inhibitor comprises at least one terpenoid. In an embodiment, the at least one terpenoid comprises a ginkgolide. In an embodiment, the at least one terpenoid comprises a bilobalide. In an embodiment, the monoamine oxidase inhibitor comprises at least one flavonoid glycoside. In an embodiment, the at least one flavonoid glycoside comprises myricetin. In an embodiment, the at least one flavonoid glycoside comprises a myricetin glycoside. In an embodiment, the at least one flavonoid glycoside comprises quercetin. In an embodiment, the at least one flavonoid glycoside comprises a quercetin glycoside.

In an embodiment, the monoamine oxidase inhibitor comprises at least one flavone glycoside. In an embodiment, the monoamine oxidase inhibitor comprises from about 10 milligrams to about 60 milligrams of flavone glycosides. In an embodiment, the monoamine oxidase inhibitor comprises at least one terpene lactone. In an embodiment, the monoamine oxidase inhibitor comprises from about 3 milligrams to about 15 milligrams of terpene lactones. In an embodiment, the monoamine oxidase inhibitor comprises about a 4 to 1 by weight ratio of flavone glycoside to terpene lactone.

In an embodiment, a pharmaceutical composition includes at least one cyclooxygenase inhibitor. In an embodiment, the cyclooxygenase inhibitor comprises a COX-2 inhibitor. In an embodiment, the cyclooxygenase inhibitor comprises a nonselective cyclooxygenase (COX) inhibitor. In an embodiment, the cyclooxygenase inhibitor comprises acetylsalicylic acid. In an embodiment, the cyclooxygenase inhibitor comprises ibuprofen. In an embodiment, the cyclooxygenase inhibitor comprises isobutylphenylpropanoic acid. In an embodiment, the cyclooxygenase inhibitor comprises (RS)-2-(4-(2-methylpropyl)phenyl)propanoic acid. In an embodiment, the cyclooxygenase inhibitor comprises a non-steroidal anti-inflammatory drug.

In an embodiment, the cyclooxygenase inhibitor comprises from about 100 milligrams to about 800 milligrams of ibuprofen. In an embodiment, the cyclooxygenase inhibitor comprises at least 100 milligrams of ibuprofen. In an embodiment, the cyclooxygenase inhibitor comprises at least 200 milligrams of ibuprofen. In an embodiment, the cyclooxygenase inhibitor comprises at least 400 milligrams of ibuprofen. In an embodiment, the cyclooxygenase inhibitor comprises at least 600 milligrams of ibuprofen. In an embodiment, the cyclooxygenase inhibitor comprises at least 700 milligrams of ibuprofen.

In an embodiment, a pharmaceutical composition includes at least one terpene. In an embodiment, the at least one terpene comprises one of borneol, β-caryophyllene, cineole, delta-3-carene, limonene, D-linalool, β-myrcene, pinene, pulegone, sabinene, or terpineol. In an embodiment, a pharmaceutical composition includes at least one terpenoid. In an embodiment, a pharmaceutical composition includes at least one terpene and at least one terpenoid.

In an embodiment, a pharmaceutical composition includes at least one nootropic agent. Non-limiting examples of nootropic agents include memory enhancers, neuro enhancers, cognitive enhancers, intelligence enhancers, and the like, or mixtures thereof. Further non-limiting examples of nootropic agents include 8-sulfocholecystokinin octapeptide, acetylcamitine, ACTH (4-7), ACTH (4-10), adafenoxate, aniracetam, cerebrolysin, choline, cytidine diphosphate choline, donepezil, ergoloid mesylates, etimizol, etiracetam, galantamine, meclofenoxate, nefiracetam, nicergoline, nicotinoyl-GABA, oxiracetam, pantogab, picamilon, piracetam, SA 4503, TA 0910, tacrine, vinpocetine, and the like, or mixtures thereof. See e.g., “NLM Controlled Vocabulary.” National Center for Biotechnology Information. U.S. National Library of Medicine, n.d. Web. 3 Jul. 2014.

In an embodiment, the at least one nootropic agent comprises a memory enhancer agent. In an embodiment, the at least one nootropic agent comprises an omega-3 fatty acid. In an embodiment, the at least one nootropic agent comprises vitamin E. In an embodiment, the at least one nootropic agent comprises one of arecoline, edrophonium, oxotremorine, deanol, or the like. In an embodiment, the at least one nootropic agent comprises a neuro enhancer agent. In an embodiment, the at least one nootropic agent comprises erythropoietin. In an embodiment, the at least one nootropic agent comprises a cognitive enhancer agent. In an embodiment, the at least one nootropic agent comprises dimebon. In an embodiment, the at least one nootropic agent comprises an intelligence enhancer agent. In an embodiment, the at least one nootropic agent comprises 8-sulfocholecystokinin octapeptide, acetylcamitine, ACTH (4-7), ACTH (4-10), adafenoxate, aniracetam, cerebrolysin, choline, cytidine diphosphate choline, donepezil, ergoloid mesylates, etimizol, etiracetam, galantamine, meclofenoxate, nefiracetam, nicergoline, nicotinoyl-GABA, oxiracetam, pantogab, picamilon, piracetam, SA 4503, TA 0910, tacrine, or vinpocetine.

In an embodiment, a pharmaceutical composition includes a pharmaceutically acceptable carrier. Non-limiting examples of pharmaceutically acceptable carrier materials include binders, disintegrating agents, extenders, fillers, humectants, lubricants, wetting agents, and the like, or mixtures thereof. Further non-limiting examples of pharmaceutically acceptable carrier materials include bentonite clay, calcium stearate, cellulose, cellulose derivatives, cetyl alcohol, citric acid, corn starch, crospovidone, glucose, glycerol monostearate, kaolin, lactose monohydrate, lactose, magnesium stearate, mannitol, microcrystalline cellulose, polyethylene glycols, polysaccharides, polyvinylpolypyrrolidone, silicic acid, sodium chloride, starch, sucrose, talc, and the like, or mixtures thereof.

In an embodiment, the pharmaceutically acceptable carrier comprises a controlled-release carrier. In an embodiment, the pharmaceutically acceptable carrier comprises a controlled-release carrier that exhibits zero order kinetics. Non-limiting examples of controlled-release carrier materials include hydrophilic materials, hydrophilic matrix materials, hydrophobic materials, hydrophobic matrix materials, and the like, or mixtures thereof. Further non-limiting of controlled-release carrier materials include polymers, protein derived materials, waxes, shellac, gums, hydrogels, oils, and the like. Further non-limiting of controlled-release carrier materials include hydrophilic matrix system including cellulosic polymers (e.g., hydroxypropylmethylcellulose, methylcellulose, hydroxypropylcellulose, hydroxyethylcellulose, sodium carboxymethylcellulose, and the like).

Further non-limiting examples of pharmaceutically acceptable carrier materials include gum/polysaccharides, polyethylene oxide, homopolymers and copolymer of acrylic acid, and the like. Further non-limiting examples of pharmaceutically acceptable carrier materials include carrageenan, chitosan, crosslinked high amylose starch, guar gum, locust bean gum, pectin, sodium alginate, xanthan gum, and the like. Further non-limiting examples of pharmaceutically acceptable carrier materials include acrylic polymers and copolymers, alkylcelluloses, alkylvinyl polymers, carboxyalkylcelluloses, cellulose ethers, methacrylic acid polymers and copolymers, and the like. Further non-limiting examples of pharmaceutically acceptable carrier materials include beeswax, carnauba wax, fatty acids, fatty alcohols, natural waxes, stearic acid, stearyl alcohol, synthetic waxes, and the like.

In an embodiment, the pharmaceutically acceptable carrier comprises a sustained release carrier. In an embodiment, the pharmaceutically acceptable carrier comprises a sustained release carrier that exhibits first order kinetics. In an embodiment, the pharmaceutically acceptable carrier comprises an immediate release carrier. In an embodiment, the pharmaceutically acceptable carrier comprises an extended release carrier. In an embodiment, the pharmaceutically acceptable carrier includes a disintegrant in an amount sufficient to cause the active agent to exhibit an immediate release profile. For example, in an embodiment, the pharmaceutically acceptable carrier includes a disintegrant in an amount sufficient to cause an active ingredient, an active compound, an active agent, or the like to exhibit an immediate release profile.

In an embodiment, the pharmaceutically acceptable carrier comprises an inhalable composition. In an embodiment, the pharmaceutically acceptable carrier comprises an ingestible composition. In an embodiment, the pharmaceutically acceptable carrier comprises a sublingual composition. In an embodiment, the pharmaceutically acceptable carrier comprises a transdermal composition. In an embodiment, the pharmaceutically acceptable carrier comprises a topical composition. In an embodiment, the pharmaceutically acceptable carrier comprises a transmucosal composition. In an embodiment, the pharmaceutically acceptable carrier comprises a rectal composition.

In an embodiment, active ingredients, active compounds, active agents, or the like can be given to a patient as such or in combination with suitable pharmaceutical excipients in the form of tablets, granules, capsules, suppositories, emulsions, suspensions, solutions, and the like.

In an embodiment, suitable carriers, solvents, gel forming ingredients, dispersion forming ingredients, antioxidants, colors, sweeteners, wetting compounds, and other ingredients normally used in this field of technology may be also used. In an embodiment, compositions containing the active ingredients, active compounds, active agents, or the like can be given via one or more routes of administration.

In an embodiment, a pharmaceutical composition includes a controlled-release carrier. In an embodiment, a pharmaceutical composition includes a sustained release carrier. In an embodiment, a pharmaceutical composition includes an immediate release carrier. In an embodiment, a pharmaceutical composition includes an extended release carrier. In an embodiment, a pharmaceutical composition includes a solid, a liquid, a solution, a suspension, a gel, a glass, or a solid dispersion.

In an embodiment, methods and compositions include a use of one or more nootropic agents in combination with at least one COX-2 inhibitor in the manufacture of a pharmaceutical formulation. For example, in an embodiment, methods and compositions include a use of one or more ginkgo biloba plant extracts in combination with at least one COX-2 inhibitor in the manufacture of a pharmaceutical formulation for use in the prevention or treatment of a condition associated with memory loss.

In an embodiment, a pharmaceutical composition includes at least one nootropic agent, at least one cyclooxygenase inhibitor, and a pharmaceutically acceptable carrier. In an embodiment, a pharmaceutical composition includes a monoamine oxidase inhibitor, at least one cyclooxygenase inhibitor, and a pharmaceutically acceptable carrier. In an embodiment, a pharmaceutical composition includes a reversible monoamine oxidase inhibitor, at least one cyclooxygenase inhibitor, and a pharmaceutically acceptable carrier. In an embodiment, a pharmaceutical composition includes a flavonoid, at least one cyclooxygenase inhibitor, and a pharmaceutically acceptable carrier. In an embodiment, a pharmaceutical composition includes at least one of apigenin, quercetin, cannflavin A, or β-sitostero, at least one cyclooxygenase inhibitor, and a pharmaceutically acceptable carrier.

In an embodiment, a pharmaceutical composition includes a flavonoid glycoside, at least one cyclooxygenase inhibitor, and a pharmaceutically acceptable carrier. In an embodiment, a pharmaceutical composition includes myricetin, at least one cyclooxygenase inhibitor, and a pharmaceutically acceptable carrier. In an embodiment, a pharmaceutical composition includes a myricetin glycoside, at least one cyclooxygenase inhibitor, and a pharmaceutically acceptable carrier. In an embodiment, a pharmaceutical composition includes quercetin, at least one cyclooxygenase inhibitor, and a pharmaceutically acceptable carrier. In an embodiment, a pharmaceutical composition includes a quercetin glycoside, at least one cyclooxygenase inhibitor, and a pharmaceutically acceptable carrier.

In an embodiment, a pharmaceutical composition includes a terpenoid, at least one cyclooxygenase inhibitor, and a pharmaceutically acceptable carrier. In an embodiment, a pharmaceutical composition includes a ginkgolide, at least one cyclooxygenase inhibitor, and a pharmaceutically acceptable carrier. In an embodiment, a pharmaceutical composition includes ginkgolide B, at least one cyclooxygenase inhibitor, and a pharmaceutically acceptable carrier. In an embodiment, a pharmaceutical composition includes ginkgolide J, at least one cyclooxygenase inhibitor, and a pharmaceutically acceptable carrier. In an embodiment, a pharmaceutical composition includes a bilobalide, at least one cyclooxygenase inhibitor, and a pharmaceutically acceptable carrier. In an embodiment, a pharmaceutical composition includes a flavone glycoside, at least one cyclooxygenase inhibitor, and a pharmaceutically acceptable carrier.

In an embodiment, active ingredients, active compounds, active agents, or the like may be administered to a patient in therapeutically effective amounts depending on age, weight, ethnic group, condition of a patient, condition to be treated, administration route, and the active ingredients, active compounds, or active agents used. For example, in an embodiment, a pharmaceutical composition includes from about 10 milligrams to about 60 milligrams of flavone glycosides, at least one cyclooxygenase inhibitor, and a pharmaceutically acceptable carrier. In an embodiment, a pharmaceutical composition includes a terpene, lactone at least one cyclooxygenase inhibitor, and a pharmaceutically acceptable carrier. In an embodiment, a pharmaceutical composition includes from about 3 milligrams to about 15 milligrams of terpene lactones, at least one cyclooxygenase inhibitor, and a pharmaceutically acceptable carrier.

In an embodiment, a pharmaceutical composition includes a ginkgo biloba extract, at least one cyclooxygenase inhibitor, and a pharmaceutically acceptable carrier.

In an embodiment, a dosage level may depend upon the activity of the particular compound, the route of administration, the severity of the condition being treated, or a condition and prior medical history of the patient being treated. In an embodiment, a pharmaceutical composition includes from about 5% by weight to about 30% by weight ginkgo flavone glycosides, from about 1% by weight to about 10% by weight ginkgolides and bilobalides, at least one cyclooxygenase inhibitor, and a pharmaceutically acceptable carrier. In an embodiment, a pharmaceutical composition includes from about 10 milligrams to about 60 milligrams of flavone glycosides, from about 3 milligrams to about 15 milligrams of terpene lactones, at least one cyclooxygenase inhibitor, and a pharmaceutically acceptable carrier.

In an embodiment, a pharmaceutical composition includes a memory enhancer agent, at least one cyclooxygenase inhibitor, and a pharmaceutically acceptable carrier. In an embodiment, a pharmaceutical composition includes a neuro enhancer agent, at least one cyclooxygenase inhibitor, and a pharmaceutically acceptable carrier. In an embodiment, a pharmaceutical composition includes erythropoietin, at least one cyclooxygenase inhibitor, and a pharmaceutically acceptable carrier. In an embodiment, a pharmaceutical composition includes a cognitive enhancer agent, at least one cyclooxygenase inhibitor, and a pharmaceutically acceptable carrier. In an embodiment, a pharmaceutical composition includes an intelligence enhancer agent, at least one cyclooxygenase inhibitor, and a pharmaceutically acceptable carrier.

In an embodiment, the at least one nootropic agent comprises 8-sulfocholecystokinin octapeptide, acetylcamitine, ACTH (4-7), ACTH (4-10), adafenoxate, aniracetam, cerebrolysin, choline, cytidine diphosphate choline, donepezil, ergoloid mesylates, etimizol, etiracetam, galantamine, meclofenoxate, nefiracetam, nicergoline, nicotinoyl-GABA, oxiracetam, pantogab, picamilon, piracetam, SA 4503, TA 0910, tacrine, or vinpocetine.

In an embodiment, a pharmaceutical composition includes one or more concentrates, extracts, resins, and the like. In an embodiment, a pharmaceutical composition includes grape skin extracts. In an embodiment, a pharmaceutical composition includes huperzia serrata extract. In an embodiment, a pharmaceutical composition includes ginkgo biloba plant extract.

In an embodiment, a pharmaceutical composition includes one or more ergogenic agents, ergogenic aids, and the like. In an embodiment, a pharmaceutical composition includes vanadium. In an embodiment, a pharmaceutical composition includes vanadyl sulfate.

In an embodiment, a pharmaceutical composition includes one or more of acetylcholinesterase inhibitors, anthocyanosides, choline, choline bitartrate, cyclohexane-1,2,3,4,5,6-hexol, dimethylaminoethanol, dimethylaminoethanol bitartrate, dimethylethanolamine, docosahexaenoic acid, ethyl apovincaminate, huperzine A, Inositol, omega-3 fatty acid, phosphatidylserine, procyanidins, vinpocetine, Vinpocetine, γ-aminobutyric acid, and the like

In an embodiment, a pharmaceutical composition includes one or more of L-glutamine, L-pyroglutamic acid, L-tyrosine, N-acetyl tyrosine, and the like.

In an embodiment, methods and compositions include a use of at least one monoamine oxidase inhibitor in combination with at least one cyclooxygenase inhibitor and a terpene in the manufacture of a pharmaceutical formulation for use in the prevention or treatment of a condition associated with memory loss. In an embodiment, a pharmaceutical composition includes a monoamine oxidase inhibitor, a cyclooxygenase inhibitor, a terpene, and a pharmaceutically acceptable carrier. In an embodiment, a pharmaceutical composition includes a nootropic agent, a cyclooxygenase inhibitor, a terpene, and a pharmaceutically acceptable carrier. In an embodiment, a pharmaceutical composition includes a terpenoid, a cyclooxygenase inhibitor, and a pharmaceutically acceptable carrier.

In an embodiment, a method of preventing or treating a condition associated with memory loss includes administering to a mammal in need thereof a therapeutically effective amount of a cyclooxygenase inhibitor in combination with a monoamine oxidase inhibitor. In an embodiment, a method of preventing or treating a condition associated with memory loss includes administering to a mammal in need thereof a nootropic agent. In an embodiment, treating may include administering to a subject, a therapeutically-effective amount of one or more compounds to prevent, control, ameliorate, eliminate, or the like, a condition associated with, for example, memory loss, a loss of cognitive ability, or the like. In an embodiment, a therapeutically effective amount, a therapeutically effective dose, or the like includes an amount of a compound associated with preventing an onset, alleviating a symptom, stopping a progression, ameliorating a condition, or the like, or resulting in another biological outcome.

In an embodiment, administering to a mammal in need thereof the therapeutically effective amount of the cyclooxygenase inhibitor in combination with the monoamine oxidase inhibitor includes administering to a mammal in need thereof a therapeutically effective amount of a cyclooxygenase-2 inhibitor in combination with the monoamine oxidase inhibitor. In an embodiment, administering to a mammal in need thereof the therapeutically effective amount of the cyclooxygenase inhibitor in combination with the monoamine oxidase inhibitor includes administering a therapeutically effective amount of a cyclooxygenase-2 inhibitor in combination with a terpenoid. In an embodiment, administering to a mammal in need thereof the therapeutically effective amount of the cyclooxygenase inhibitor in combination with the monoamine oxidase inhibitor includes administering a therapeutically effective amount of ibuprofen in combination with the monoamine oxidase inhibitor.

In an embodiment, a method of treating, preventing, or ameliorating one or more symptoms associated with memory loss includes administering to a mammal in need thereof a pharmaceutically active amount of a cyclooxygenase inhibitor in combination with a nootropic agent.

In an embodiment, a pharmaceutically active amount includes an amount that will lead to the desired pharmacological effects, therapeutic effects, or the like. In an embodiment, a pharmaceutically active amount includes an amount of the combination product that is effective to achieve its intended purpose. In an embodiment, a dosage regimen for treating a condition with a combination product can be selected in accordance with a variety of factors, including the type, age, weight, sex, diet and medical condition of the patient. In an embodiment, a pharmaceutical composition includes one or more active ingredients in any form suitable to be used for a medical purpose. In an embodiment, a treatment includes any therapeutic application that can benefit a human or non-human mammal. In an embodiment, a treatment may include treating an existing condition or it may be prophylactic.

In an embodiment, administering to the mammal in need thereof the pharmaceutically active amount of the cyclooxygenase inhibitor in combination with the nootropic agent includes administering the therapeutically effective amount of the cyclooxygenase inhibitor in combination with one or more of 8-sulfocholecystokinin octapeptide, acetylcamitine, ACTH (4-7), ACTH (4-10), adafenoxate, aniracetam, cerebrolysin, choline, cytidine diphosphate choline, donepezil, ergoloid mesylates, etimizol, etiracetam, galantamine, meclofenoxate, nefiracetam, nicergoline, nicotinoyl-GABA, oxiracetam, pantogab, picamilon, piracetam, SA 4503, TA 0910, tacrine, or vinpocetine. In an embodiment, administering to the mammal in need thereof the pharmaceutically active amount of the cyclooxygenase inhibitor in combination with the nootropic agent includes administering to a mammal in need thereof a pharmaceutically active amount of isobutylphenylpropanoic acid in combination with the nootropic agent. In an embodiment, administering to the mammal in need thereof the pharmaceutically active amount of the cyclooxygenase inhibitor in combination with the nootropic agent includes administering to a mammal in need thereof a pharmaceutically active amount of (RS)-2-(4-(2-methylpropyl)phenyl)propanoic acid in combination with at least one of a ginkgolide or a bilobalide.

In an embodiment, a method of preventing or treating a condition associated with a loss of cognitive ability in a human subject includes administering to a subject in need thereof a therapeutically effective amount of a cyclooxygenase inhibitor in combination with at least one terpene and at least one terpenoid. In an embodiment, administering to the subject in need thereof the therapeutically effective amount of the cyclooxygenase inhibitor in combination with the at least one terpene includes administering the therapeutically effective amount of (RS)-2-(4-(2-methylpropyl)phenyl)propanoic acid in combination with at least one of borneol, β-caryophyllene, cineole, delta-3-carene, limonene, D-linalool, β-myrcene, pinene, pulegone, sabinene, or terpineol, and at least one terpenoid. In an embodiment, administering to the subject in need thereof the therapeutically effective amount of the cyclooxygenase inhibitor in combination with the at least one terpene includes administering the therapeutically effective amount of ibuprofen in combination with at least one of terpene, and at least one of a ginkgolide or a bilobalide.

In an embodiment, the method of preventing or treating a condition associated with a loss of cognitive ability in a human subject includes administering to a subject in need thereof a therapeutically effective amount of a nootropic agent. In an embodiment, administering to a subject in need thereof a therapeutically effective amount of the nootropic agent includes administering to a subject in need thereof a therapeutically effective amount of 8-sulfocholecystokinin octapeptide, acetylcamitine, ACTH (4-7), ACTH (4-10), adafenoxate, aniracetam, cerebrolysin, choline, cytidine diphosphate choline, donepezil, ergoloid mesylates, etimizol, etiracetam, galantamine, meclofenoxate, nefiracetam, nicergoline, nicotinoyl-GABA, oxiracetam, pantogab, picamilon, piracetam, SA 4503, TA 0910, tacrine, or vinpocetine.

While particular aspects of the present subject matter described herein have been shown and described, it will be apparent to the reader that, based upon the teachings herein, changes and modifications can be made without departing from the subject matter described herein and its broader aspects and, therefore, the appended claims are to encompass within their scope all such changes and modifications as are within the true spirit and scope of the subject matter described herein. In general, terms used herein, and especially in the appended claims (e.g., bodies of the appended claims) are generally intended as “open” terms (e.g., the term “including” should be interpreted as “including but not limited to,” the term “having” should be interpreted as “having at least,” the term “includes” should be interpreted as “includes but is not limited to,” etc.). Further, if a specific number of an introduced claim recitation is intended, such an intent will be explicitly recited in the claim, and in the absence of such recitation no such intent is present. For example, as an aid to understanding, the following appended claims may contain usage of the introductory phrases “at least one” and “one or more” to introduce claim recitations. However, the use of such phrases should not be construed to imply that the introduction of a claim recitation by the indefinite articles “a” or “an” limits any particular claim containing such introduced claim recitation to claims containing only one such recitation, even when the same claim includes the introductory phrases “one or more” or “at least one” and indefinite articles such as “a” or “an” (e.g., “a” and/or “an” should typically be interpreted to mean “at least one” or “one or more”); the same holds true for the use of definite articles used to introduce claim recitations. In addition, even if a specific number of an introduced claim recitation is explicitly recited, such recitation should typically be interpreted to mean at least the recited number (e.g., the bare recitation of “two recitations,” without other modifiers, typically means at least two recitations, or two or more recitations). Furthermore, in those instances where a convention analogous to “at least one of A, B, and C, etc.” is used, in general such a construction is intended in the sense of the convention (e.g., “a system having at least one of A, B, and C” would include but not be limited to systems that have A alone, B alone, C alone, A and B together, A and C together, B and C together, and/or A, B, and C together, etc.). In those instances where a convention analogous to “at least one of A, B, or C, etc.” is used, in general such a construction is intended in the sense of the convention (e.g., “a system having at least one of A, B, or C” would include but not be limited to systems that have A alone, B alone, C alone, A and B together, A and C together, B and C together, and/or A, B, and C together, etc.). Typically a disjunctive word or phrase presenting two or more alternative terms, whether in the description, claims, or drawings, should be understood to contemplate the possibilities of including one of the terms, either of the terms, or both terms unless context dictates otherwise. For example, the phrase “A or B” will be typically understood to include the possibilities of “A” or “B” or “A and B.”

With respect to the appended claims, the operations recited therein generally may be performed in any order. Also, although various operational flows are presented in a sequence(s), it should be understood that the various operations may be performed in orders other than those that are illustrated, or may be performed concurrently. Examples of such alternate orderings includes overlapping, interleaved, interrupted, reordered, incremental, preparatory, supplemental, simultaneous, reverse, or other variant orderings, unless context dictates otherwise. Furthermore, terms like “responsive to,” “related to,” or other past-tense adjectives are generally not intended to exclude such variants, unless context dictates otherwise.

U.S. Application Nos. 62/027,391 and 62/027,374 are incorporated herein by reference in their entireties. In addition, the embodiments, features, systems, devices, materials, methods, and techniques described herein may, in certain embodiments, be applied to or used in connection with any one or more of the embodiments, features, systems, devices, compositions, materials, methods, and techniques disclosed in the above-mentioned U.S. Application Nos. 62/027,391 and 62/027,374. At least some embodiments disclosed herein can be contained in packaging disclosed in U.S. Provisional Patent Application No. 62/027,391. In some embodiments, the packaging includes sealed compartments for holding the same or different units of medication disclosed herein. A user can selectively open compartments to access each dose. For example, a unit dose package can be a blister pack with backing member and a blister mounted defining individual cavities for storing doses medication. Each cavity can contain a single dose, such a pill. In some embodiments, the packaging includes single or multi-dose unit dose packages in the form of packets, pouches, or the like. Other types of packaging can be used can be include closures, lids, break-away member, cavities, or combinations thereof and may be reusable (e.g., reclosable) or single use (e.g., non-reclosable). Unit dose packaging may include any desired number of doses and may include tamper evidence, child-resistance features and may include labeling with, for example, dose information, expiration dates, machine readable information (e.g., barcodes), or other information.

While various aspects and embodiments have been disclosed herein, other aspects and embodiments are contemplated. The various aspects and embodiments disclosed herein are for purposes of illustration and are not intended to be limiting, with the true scope and spirit being indicated by the following claims.

1. A pharmaceutical composition, comprising:
a monoamine oxidase inhibitor,
a cyclooxygenase inhibitor; and
a pharmaceutically acceptable carrier.

2. (canceled)

3. The pharmaceutical composition of claim 1, wherein the monoamine oxidase inhibitor comprises at least one flavonoid.

4. (canceled)

5. The pharmaceutical composition of claim 1, wherein the monoamine oxidase inhibitor comprises at least one flavonoid glycoside.

6. (canceled)

7. (canceled)

8. (canceled)

9. (canceled)

10. The pharmaceutical composition of claim 1, wherein the monoamine oxidase inhibitor comprises at least one terpenoid.

11. (canceled)

12. (canceled)

13. (canceled)

14. (canceled)

15. (canceled)

16. (canceled)

17. (canceled)

18. The pharmaceutical composition of claim 1, wherein the cyclooxygenase inhibitor comprises a cyclooxygenase-2 inhibitor.

19. (canceled)

20. (canceled)

21. The pharmaceutical composition of claim 1, wherein the cyclooxygenase inhibitor comprises ibuprofen.

22. (canceled)

23. (canceled)

24. (canceled)

25. (canceled)

26. (canceled)

27. (canceled)

28. (canceled)

29. (canceled)

30. The pharmaceutical composition of claim 1, wherein the cyclooxygenase inhibitor comprises a non-steroidal anti-inflammatory drug.

31. The pharmaceutical composition of claim 1, comprising: at least one terpene.

32. (canceled)

33. The pharmaceutical composition of claim 1, comprising: at least one terpenoid.

34. (canceled)

35. The pharmaceutical composition of claim 1, comprising: at least one nootropic agent.

36. (canceled)

37. (canceled)

38. (canceled)

39. (canceled)

40. (canceled)

41. (canceled)

42. The pharmaceutical composition of claim 35, wherein the at least one nootropic agent comprises a cognitive enhancer agent.

43. (canceled)

44. (canceled)

45. (canceled)

46. (canceled)

47. (canceled)

48. (canceled)

49. (canceled)

50. (canceled)

51. A pharmaceutical composition, comprising:
at least one nootropic agent,
at least one cyclooxygenase inhibitor; and
a pharmaceutically acceptable carrier.

52. The pharmaceutical composition of claim 51, wherein the at least one nootropic agent comprises a monoamine oxidase inhibitor.

53. (canceled)

54. The pharmaceutical composition of claim 51, wherein the at least one nootropic agent comprises a flavonoid.

55. (canceled)

56. (canceled)

57. The pharmaceutical composition of claim 51, wherein the at least one nootropic agent comprises myricetin.

58. (canceled)

59. (canceled)

60. (canceled)

61. (canceled)

62. The pharmaceutical composition of claim 51, wherein the at least one nootropic agent comprises a ginkgolide.

63. (canceled)

64. (canceled)

65. The pharmaceutical composition of claim 51, wherein the at least one nootropic agent comprises a bilobalide.

66. (canceled)

67. (canceled)

68. The pharmaceutical composition of claim 51, wherein the at least one nootropic agent comprises a terpene lactone.

69. (canceled)

70. The pharmaceutical composition of claim 51, wherein the at least one nootropic agent comprises a ginkgo biloba extract.

71. The pharmaceutical composition of claim 51, wherein the at least one nootropic agent comprises a ginkgo biloba extract composition including from about 5% by weight to about 30% by weight ginkgo flavone glycosides, and from about 1% by weight to about 10% by weight ginkgolides and bilobalides.

72. (canceled)

73. The pharmaceutical composition of claim 51, wherein the at least one nootropic agent comprises a memory enhancer agent.

74. (canceled)

75. (canceled)

76. The pharmaceutical composition of claim 51, wherein the at least one nootropic agent comprises a cognitive enhancer agent.

77. (canceled)

78. (canceled)

79. (canceled)

80. (canceled)

81. A method of preventing or treating a condition associated with memory loss, comprising:
administering to a mammal in need thereof a therapeutically effective amount of a cyclooxygenase inhibitor in combination with a monoamine oxidase inhibitor.

82. The method of preventing or treating a condition associated with memory loss of claim 81, wherein administering to a mammal in need thereof the therapeutically effective amount of the cyclooxygenase inhibitor in combination with the monoamine oxidase inhibitor includes administering to a mammal in need thereof a therapeutically effective amount of a cyclooxygenase-2 inhibitor in combination with the monoamine oxidase inhibitor.

83. The method of preventing or treating a condition associated with memory loss of claim 81, wherein administering to a mammal in need thereof the therapeutically effective amount of the cyclooxygenase inhibitor in combination with the monoamine oxidase inhibitor includes administering a therapeutically effective amount of a cyclooxygenase-2 inhibitor in combination with a terpenoid.

84. The method of preventing or treating a condition associated with memory loss of claim 81, wherein administering to a mammal in need thereof the therapeutically effective amount of the cyclooxygenase inhibitor in combination with the monoamine oxidase inhibitor includes administering a therapeutically effective amount of ibuprofen in combination with the monoamine oxidase inhibitor.

85. (canceled)

86. (canceled)

87. (canceled)

88. (canceled)

89. (canceled)

90. A method of preventing or treating a condition associated with a loss of cognitive ability in a human subject, comprising:
administering to a subject in need thereof a therapeutically effective amount of a cyclooxygenase inhibitor in combination with at least one terpene and at least one terpenoid.

91. The method of preventing or treating a condition associated with a loss of cognitive ability in a human subject of claim 90, wherein administering to the subject in need thereof the therapeutically effective amount of the cyclooxygenase inhibitor in combination with the at least one terpene includes administering the therapeutically effective amount of (RS)-2-(4-(2-methylpropyl)phenyl)propanoic acid in combination with at least one of a borneol, β-caryophyllene, cineole, delta-3-carene, limonene, D-linalool, β-myrcene, pinene, pulegone, sabinene, or terpineol, and at least one terpenoid.

92. The method of preventing or treating a condition associated with a loss of cognitive ability in a human subject of claim 90, wherein administering to the subject in need thereof the therapeutically effective amount of the cyclooxygenase inhibitor in combination with the at least one terpene includes administering the therapeutically effective amount of ibuprofen in combination with at least one of terpene, and at least one of a ginkgolide or a bilobalide.

93. The method of preventing or treating a condition associated with a loss of cognitive ability in a human subject of claim 90, comprising:
administering to a subject in need thereof a therapeutically effective amount of a nootropic agent.

94. The method of preventing or treating a condition associated with a loss of cognitive ability in a human subject of claim 93, wherein administering to a subject in need thereof a therapeutically effective amount of the nootropic agent includes administering to a subject in need thereof a therapeutically effective amount of 8-sulfocholecystokinin octapeptide, acetylcamitine, ACTH (4-7), ACTH (4-10), adafenoxate, aniracetam, cerebrolysin, choline, cytidine diphosphate choline, donepezil, ergoloid mesylates, etimizol, etiracetam, galantamine, meclofenoxate, nefiracetam, nicergoline, nicotinoyl-GABA, oxiracetam, pantogab, picamilon, piracetam, SA 4503, TA 0910, tacrine, or vinpocetine.
